(19) United States
(12) Patent Application Publication
(10) Pub. No.: US 2015/0111929 A1
(43) Pub. Date: Apr. 23, 2015

(54) PROCESS FOR PREPARING CRYSTALLINE SORAFENIB TOSYLATE

(71) Applicant: SHILPA MEDICARE LIMITED, RAICHUR, OT (IN)

(72) Inventors: Prashant Purohit, Vizianagaram (IN); Sriram Rampalli, Vizianagaram (IN); Mohanrao Seshagiri Vijaya Murali, Vizianagaram (IN); Lavkumar Upalla, Vizianagaram (IN)

(21) Appl. No.: 14/381,226

(22) PCT Filed: Dec. 31, 2012
(86) PCT No.: PCT/IN2012/000866

§ 371 Date: Aug. 27, 2014

(30) Foreign Application Priority Data
May 23, 2012 (IN) 2059/CHE/2012

(51) Int. Cl. C07D 213/81

(52) U.S. Cl. CPC C07D 213/81
USPC 514/350; 546/298

(57) ABSTRACT

The present invention provides an industrially suitable process for the preparation of substantially pure 4-{4-[({[4-chloro-3-(trifluoromethyl)-phenyl]amino}carbonyl)amino]phenoxy}-N-methylpyridine-2-carboxamide or Sorafenib and its tosylate salt, with a suitable impurity profile and without requirement of any additional purification steps. The present invention also provides Sorafenib base (II) as stable crystalline Form-SSB.

The present invention further relates to a process for the preparation of crystalline Sorafenib tosylate Form-I which is free from contamination of any other polymorphic form of Sorafenib tosylate, for e.g. Form II or Form III, and does not involve any seeding requirement for crystallization step.

### FIELD OF INVENTION

The present invention provides an industrially suitable process for the preparation of substantially pure 4-{4-[({[4-chloro-3-(trifluoromethyl)-phenyl]amino}carbonyl)amino]phenoxy}-N-methylpyridine-2-carboxamide or Sorafenib and its tosylate salt, with a suitable impurity profile and without requirement of any additional purification steps. The present invention also provides Sorafenib base (II) as a stable crystalline Form-SSB.

The present invention further relates to a process for the preparation of crystalline Sorafenib tosylate Form-I which is free from contamination of any other polymorphic form of Sorafenib tosylate, for e.g. Form II or Form III, and does not involve any seeding requirement for crystallization step.

### BACKGROUND OF THE INVENTION

Sorafenib is chemically also known as 4-{4-[({([4-chloro-3-(trifluoromethyl)-phenyl]amino}carbonyl)amino]phenoxy}-N-methylpyridine-2-carboxamide (II).

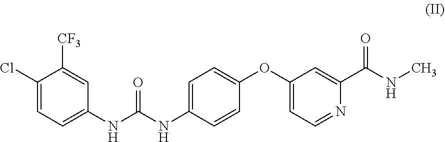 [SMILES: CNC(=O)c1cc(Oc2ccc(NC(=O)Nc3ccc(Cl)c(C(F)(F)F)c3)cc2)ccn1]

Sorafenib blocks the enzyme RAF kinase, a critical component of the RAF/MEK/ERK signaling pathway that controls cell division and proliferation. In addition, Sorafenib inhibits the VEGFR-2/PDGFR-beta signaling cascade, thereby blocking tumor angiogenesis. It is a small molecular inhibitor of RAF kinase, PDGF (platelet-derived growth factor), VEGF receptor2 and 3 and c Kit, the receptor for stem cell factor.

Sorafenib, marketed as NEXAVAR® by Bayer, is a drug approved for the treatment of advanced renal cell carcinoma (primary kidney cancer) and advanced hepatocellular carcinoma (primary liver cancer). NEXAVAR, is the tosylate salt of Sorafenib (I). In the label of USFDA, NEXAVAR is chemically mentioned as 4-(4-{3-[4-Chloro-3-(trifluoromethyl)phenyl]ureido}phenoxy)N2methylpyridine-2-carboxamide 4-methylbenzenesulfonate i.e. Sorafenib tosylate. Sorafenib tosylate is a white to yellowish or brownish solid which is practically insoluble in aqueous media and slightly soluble in ethanol.

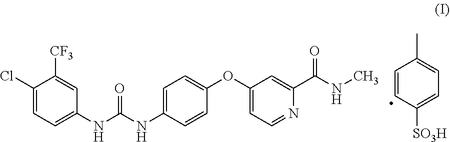 [SMILES: CNC(=O)c1cc(Oc2ccc(NC(=O)Nc3ccc(Cl)c(C(F)(F)F)c3)cc2)ccn1 ; Cc1ccc(S(=O)(=O)O)cc1]

The compound 4-{4-[({[4-chloro-3-(trifluoromethyl)-phenyl]amino}carbonyl)amino]phenoxy}-N-methylpyridine-2-carboxamide or Sorafenib of formula II is described in WO 00/42012. WO 00/42012 also reports a process for the preparation of Sorafenib base and its analogues. Pharmaceutically acceptable salts of Sorafenib, particularly Sorafenib tosylate of formula I are mentioned in patent application references WO 00/42012, WO 03/068228 and WO 03/047579.

Bankston et al. in Organic Process Research & Development, 2002, 6, 777-781 discloses a process for the preparation of Sorafenib tosylate, which involves reacting 2-picolinic acid with thionyl chloride in a solvent inert towards thionyl chloride without using dimethylformamide to form acid chloride salt. This acid salt on further reaction with aqueous solution of methylamine or gaseous methyl amine gives amide derivative, which on reaction with 4-aminophenol along with addition of carbonate salt in the presence of base yields 4-(4-aminophenoxy)-N-methylpicolinamide-4-(4-aminophenoxy)-N-methylpicolinamide which when reacted with 4-chloro-3-(trifluoromethyl) phenyl isocyanate in a non-chlorinated organic solvent, inert towards isocyanate gives Sorafenib. Sorafenib by admixing with p-toluenesulfonic acid in a polar solvent gives Sorafenib tosylate.

Patent application WO 2006/034796 discloses a process for the preparation of Sorafenib tosylate, which involves reacting 4-(4-aminophenoxy)-N-methylpicolinamide with 4-chloro-3(trifluoromethyl)phenyl isocyanate in a non-chlorinated organic solvent, giving Sorafenib base. After admixing the Sorafenib base with p-toluene sulfonic acid in polar solvent, water is added to the reaction mixture and, if appropriate, a clarifying filtration is conducted and if required seeding procedure is done to give Sorafenib tosylate.

WO 2006/034797 provides a process for the preparation of Polymorph I of Sorafenib tosylate, which involves the initial preparation of polymorph II of Sorafenib tosylate and its further conversion to Polymorph I of Sorafenib tosylate.

Patent application WO 2011/036647 discloses a process for the preparation of Polymorph I of Sorafenib tosylate, which involves the addition of p-toluenesulfonic acid to Sorafenib in the presence of water.

Though the review of the above mentioned literature discloses many processes for the preparation of Sorafenib and its tosylate salt, but due to one or more reasons, for e.g. unfavorable impurity profile, multiple steps, use of multiple solvents, low yield and difficult isolation procedures etc. these processes are not particularly convenient and suitable for industrial scale production.

Thus, there is an apparent need to develop improved processes for the preparation of highly pure Sorafenib and its tosylate salt, which may be cost-effective, industrially amenable, with good % of yield and may overcome the drawbacks of various prior disclosed processes. Thus according to the present invention there is provided a novel industrially applicable process for the preparation of Sorafenib and its tosylate salt, with suitable impurity profile and without requirement of any additional purification steps.

For Sorafenib base, subsequent to process, the concern has remained for the solid form isolated. Though extensive information is available for polymorphic forms of Sorafenib tosylate, not much is known regarding polymorphism in actual active moiety of the drug i.e. Sorafenib base. Patent applications WO2009/106825 and WO 2010/142678 disclose Sorafenib base amorphous form.

Existence of polymorphism is known to be unique phenomenon in solid materials, wherein existence of different physical forms including shape, size, and arrangement of molecules in the physical state or polymorphs of same compound are known in the nature. A single compound, or a salt complex, may give rise to a variety of solids having distinct physical properties, which often results in substantial differences in bioavailability, stability, and other differences between production lots of formulated pharmaceutical products. Due to this reason, since polymorphic forms can vary in their chemical and physical properties, regulatory authorities often require that efforts be made to identify all polymorphic forms, e.g., hydrate or anhydrate, crystalline or amorphous, solvated or un-solvated forms, base forms or salt forms etc. of the drug substances. However, the existence, and possible numbers, of polymorphic forms for a given compound cannot be predicted. In addition, there are no “standard” procedures that can be used to prepare polymorphic forms of a substance.

New polymorphs of pharmaceutically active/useful compounds provide an opportunity to improve the drug performance characteristics of such product. Further, discovery of polymorphic forms may help in the identification of the polymorphic content of a batch of an active pharmaceutical ingredient. Therefore, inventors of the present application provide new stable crystalline form of Sorafenib base designated as Form-SSB. Crystalline Sorafenib base Form-SSB is very stable, wherein the physicochemical properties remain substantially the same (at least up to more than 6 months). This stable form offers various advantages in terms of storage, shelf life and favorable impurity profile.

### SUMMARY OF THE INVENTION

Particular aspects of the present application relate to the process for the preparation of pure 4-{4-[({[4-chloro-3-(trifluoromethyl)-phenyl]amino}carbonyl)amino]phenoxy}-N-methyl pyridine-2-carboxamide or Sorafenib and its tosylate salt.

In one aspect of the present application, the present invention provides a process for the preparation of Sorafenib comprising, reaction of 4-(4-aminophenoxy)-N-methylpicolinamide (III) with 4-chloro-3-(trifluoromethyl)phenylisocyanate (IV) in a high boiling organic solvent at a temperature ranging between 75-90° C., to yield Sorafenib base (II).

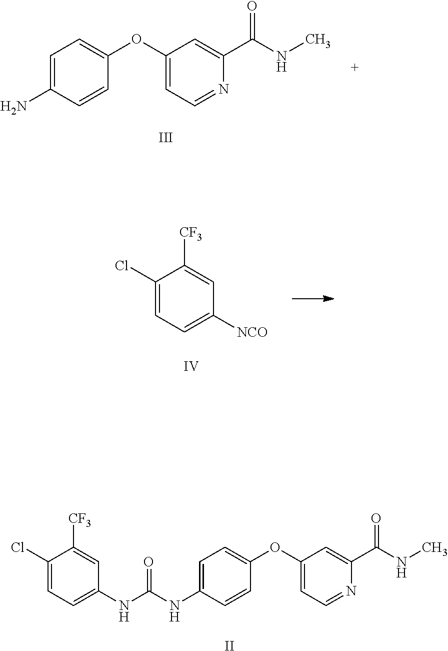

In a further aspect of the present application, the present invention provides Sorafenib base-Crystalline Form-SSB characterized by X-ray powder diffraction pattern comprising at least 5 characteristic 2θ° peaks selected from the XRPD peak set of 9.9, 11.4, 12.6, 14.6, 15.2, 15.6, 18.1, 18.6, 21.8, 22.5, 22.9, 23.6, 24.8, 25.2 0.20 2θ° and DSC isotherm comprising a single endothermic peak ranging between 202 to 212° C.

In another aspect of the present application, the present invention provides a process for the preparation of Sorafenib tosylate salt, comprising steps of:
a) reacting 4-(4-aminophenoxy)-N-methylpicolinamide (III) with 4-chloro-3-(trifluoromethyl)phenylisocyanate (IV) in a high boiling organic solvent at a temperature ranging between 75-90° C., to yield Sorafenib base (II);

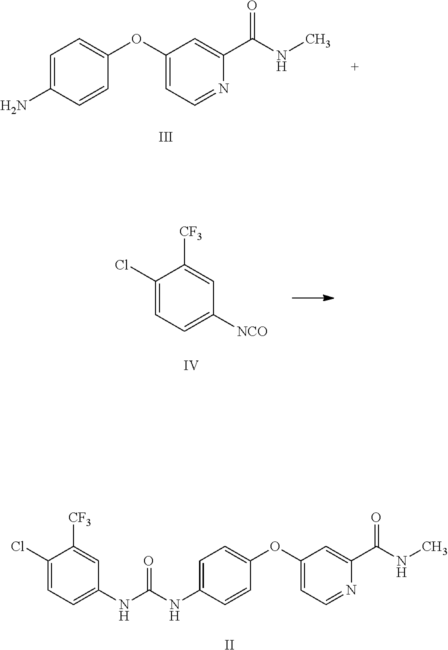

b) combining Sorafenib base (II) with p-toluenesulfonic acid in the presence of methyl ethyl ketone at a temperature below 35° C., to give crystalline Sorafenib tosylate (I).

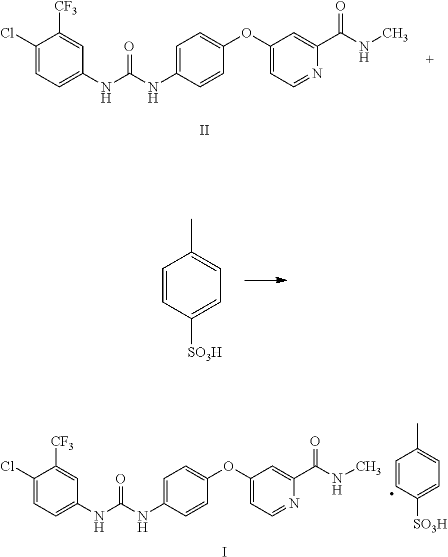

In another aspect of the present application, the present invention provides a one-pot process for the preparation of Sorafenib tosylate (I), comprising the steps of:
a) reacting 4-(4-aminophenoxy)-N-methylpicolinamide (III) with 4-chloro-3-(trifluoromethyl)phenylisocyanate (IV) in a high boiling organic solvent at a temperature ranging between 75-90° C.;

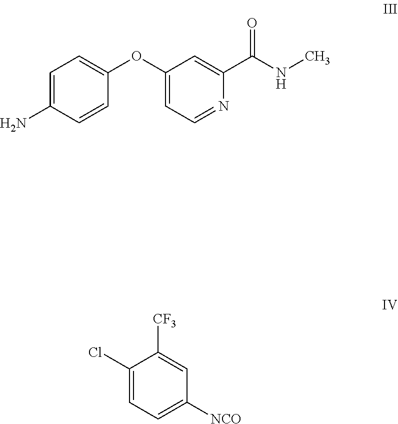

b) adding p-toluenesulfonic acid to the reaction mass of step a) in the presence of methyl ethyl ketone at a temperature below 35° C.;
c) isolating the material as Sorafenib tosylate (I).

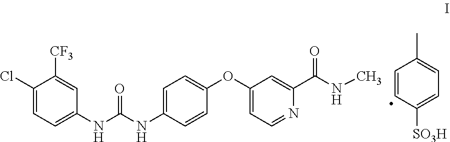

In a further aspect of the present application, the present invention provides process for the preparation of Sorafenib tosylate, wherein Sorafenib tosylate obtained is substantially pure crystalline Sorafenib tosylate Form I which is free from contamination of any other polymorphic form of Sorafenib tosylate, for e.g. Form II or Form III.

Further particular aspects of the invention are detailed in the description part of the specification, wherever appropriate.

### BRIEF DESCRIPTION OF THE DRAWINGS

FIG. 1: PXRD pattern of crystalline Sorafenib base (Form-SSB) obtained according to the procedure given in Example 1.

FIG. 2: DSC curve of crystalline Sorafenib base (Form-SSB) obtained according to the procedure given in Example 1.

FIG. 3: TGA thermogram of crystalline Sorafenib base (Form-SSB) obtained according to the procedure given in Example 1.

FIG. 4: PXRD pattern of crystalline Sorafenib tosylate obtained according to the procedure given in Example 2.

FIG. 5: DSC curve of crystalline Sorafenib tosylate obtained according to the procedure given in Example 2.

FIG. 6: TGA spectrum of crystalline Sorafenib tosylate obtained according to the procedure given in Example 2.

### DETAILED DESCRIPTION

As set forth herein embodiments of the present invention provide the process for preparation of Sorafenib and its tosylate salt, to give substantially pure product with a suitable impurity profile and without requirement of any additional purification steps.

One embodiment of the present application provides a process for the preparation of Sorafenib, comprising, reaction of 4-(4-aminophenoxy)-N-methylpicolinamide (III) with 4-chloro-3-(trifluoromethyl)phenylisocyanate (IV) in a high boiling organic solvent at a temperature ranging between 75-90° C., to yield Sorafenib base (II).

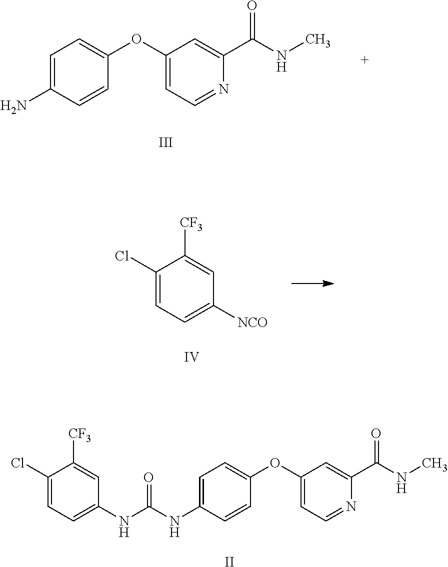

High boiling organic solvent used in this reaction may be selected from C₄₋₈ ketones or a mixture thereof. Non-limiting examples of the C₄₋₈ ketones that can be used as high boiling organic solvent in this reaction include methyl ethyl ketone (MEK) or methyl isobutyl ketone (MIBK) or a mixture thereof.

Reaction temperature plays a very important role in this reaction with regard to the purity of the obtained final product. Reaction of 4-(4-aminophenoxy)-N-methylpicolinamide (III) with 4-chloro-3-(trifluoromethyl)phenylisocyanate (IV), is carried out at high temperature ranging from 75-90° C., more preferably 80-85° C. but not at temperature lower than 70° C.

It was observed that at lower temperature for e.g. below 70° C., a substantial amount of the starting material 4-(4-aminophenoxy)-N-methylpicolinamide (III) remains unreacted and is as such carried till the final API stage as a residual impurity. This impurity 4-(4-aminophenoxy)-N-methylpicolinamide (III) has been found to be mutagenic and is very difficult to remove by purification of the final API.

High temperature conditions employed in this reaction of the present invention provide the advantage that the amount of unreacted 4-(4-aminophenoxy)-N-methylpicolinamide found in the final API is very well below the stipulated regulatory requirement of not more than 0.1%.

The quantity of the high boiling solvent used in this reaction is also pivotal in the reaction course. The quantity of high boiling solvent ranges between 2 to 5 times (in volume) with respect to the weight of 4-(4-aminophenoxy)-N-methylpicolinamide. In one particular embodiment, for 20 g of 4-(4-aminophenoxy)-N-methylpicolinamide, 60 ml of the high boiling solvent methyl ethyl ketone was used.

The reaction of 4-(4-aminophenoxy)-N-methylpicolinamide (III) with 4-chloro-3-(trifluoromethyl)phenylisocyanate (IV) is done by stirring the reaction mass at the raised temperatures mentioned, for time ranging between 2-8 hrs (depending on the actual reaction conditions employed).

Optionally, the Sorafenib base as obtained in the present reaction may also be given washings with toluene or a high boiling organic solvent used initially, at temperature ranging between 20-70° C., After drying of the reaction mass at temperature ranging between 45-60° C., isolation of the product is done by conventional methods which include but are not limited to cooling the reaction mass, filtering (with or without vacuum) and neutralizing wherever required.

In another embodiment of the present application, the present invention provides Sorafenib base as Crystalline Form-SSB, which is characterized by X-ray powder diffraction pattern comprising at least 5 characteristic 2θ° peaks selected from the XRPD peak set of 9.9, 11.4, 12.6, 14.6, 15.2, 15.6, 18.1, 18.6, 21.8, 22.5, 22.9, 23.6, 24.8, 25.2±0.20 2θ° and DSC isotherm comprising a single endothermic peak ranging between 202 to 212° C.

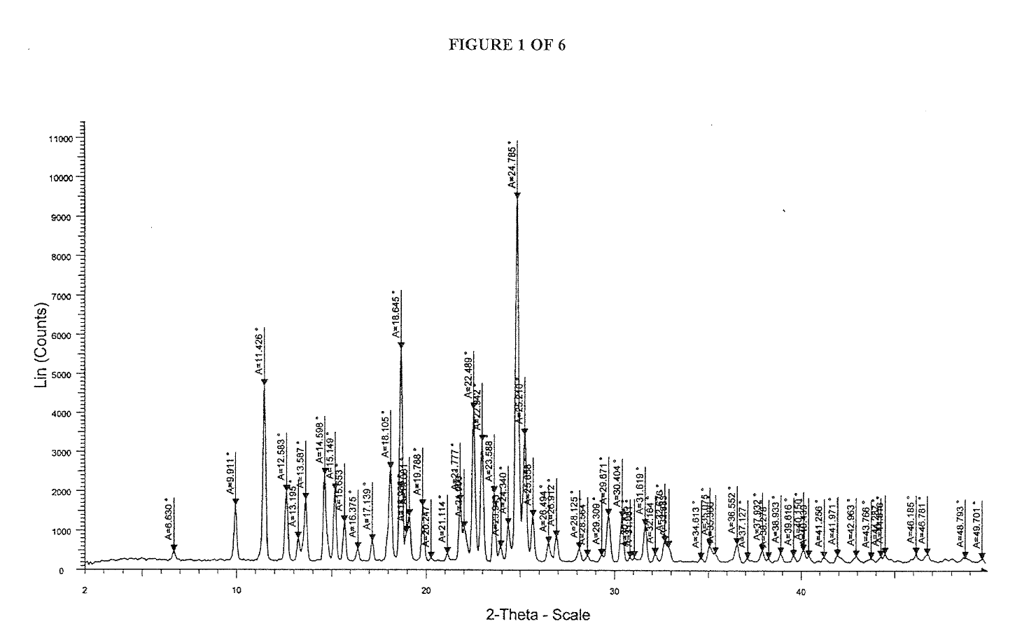

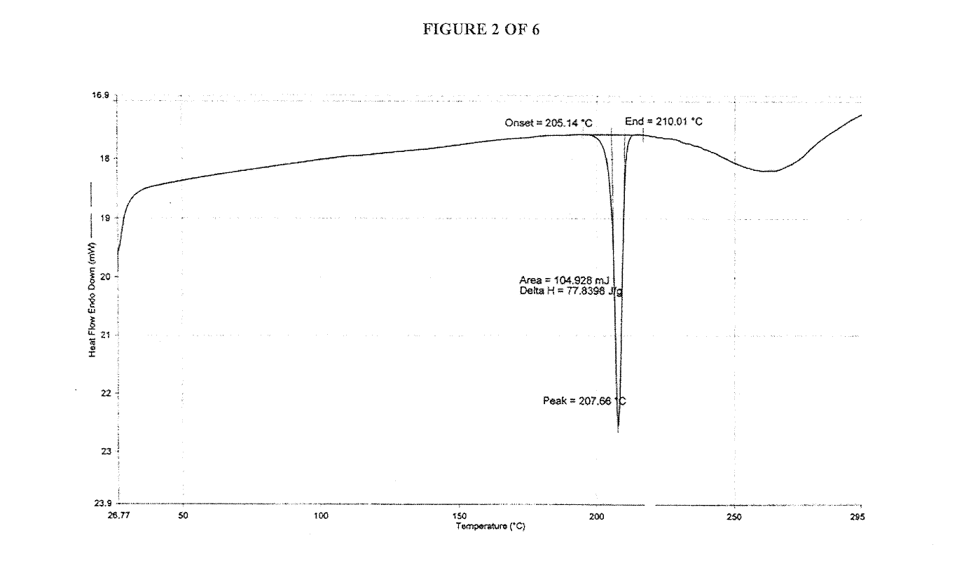

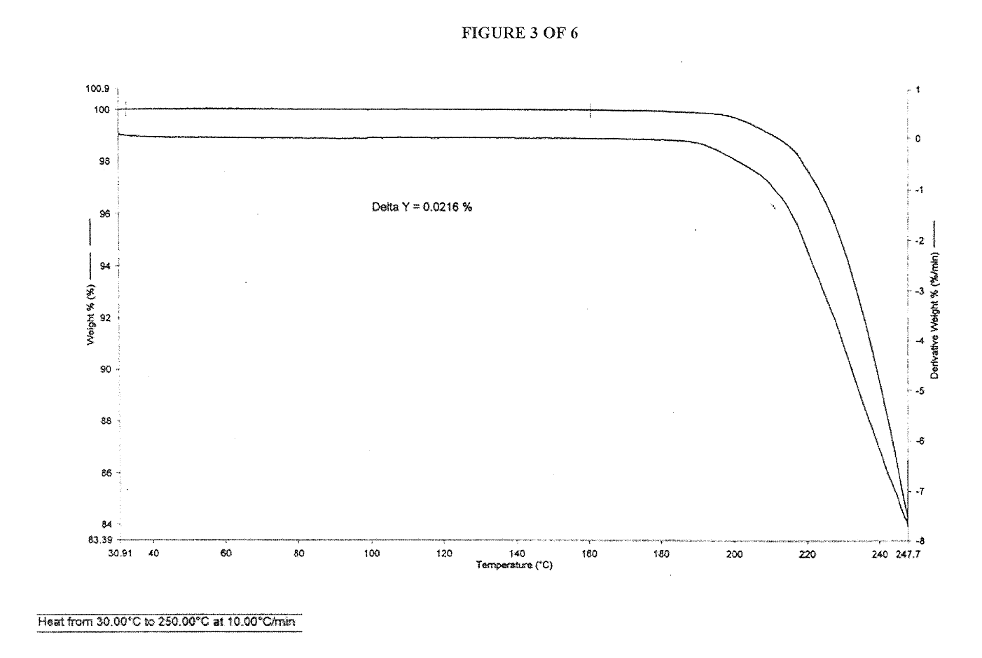

Sorafenib base crystalline Form-SSB, according to the present invention is characterized by X-ray powder diffraction pattern substantially according to FIG. 1, DSC isothermal pattern substantially according to FIG. 2 and TGA thermogram substantially according to FIG. 3. The characteristic peaks and their d spacing values of the new crystalline Form-SSB are tabulated in the Table-1.

TABLE 1
Characteristic XRPD Peaks of Sorafenib
base Crystalline Form-SSB

| S. No. | Angle (2θ°) ± 0.20 | d Spacing Value (A°) |
|---|---|---|
| 1. | 9.90 | 8.917 |
| 2. | 11.42 | 7.738 |
| 3. | 12.58 | 7.029 |
| 4. | 14.60 | 6.603 |
| 5. | 15.20 | 5.844 |
| 6. | 15.65 | 5.657 |
| 7. | 18.11 | 4.896 |
| 8. | 18.65 | 4.755 |
| 9. | 21.78 | 4.078 |
| 10. | 22.49 | 3.950 |
| 11. | 22.94 | 3.873 |
| 12. | 23.59 | 3.769 |
| 13. | 24.78 | 3.590 |
| 14. | 25.21 | 3.530 |

Minor variations in the observed 2θ° angles values may be expected based on the analyst person, the specific XRPD diffractometer employed and the sample preparation technique. Further possible variations may also be expected for the relative peak intensities, which may be largely affected by the non-uniformity of the particle size of the sample. Hence, identification of the exact crystalline form of a compound should be based primarily on observed 2 theta angles with lesser importance attributed to relative peak intensities. The 2 theta diffraction angles and corresponding d-spacing values account for positions of various peaks in the X-ray powder diffraction pattern. D-spacing values are calculated with observed 2 theta angles and copper K a wavelength using the Bragg equation well known to those of having skill in the art of XRPD diffractometry science.

In view of possibility of marginal error in the assigning 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of Sorafenib base over FIG. 1 and readily determine whether the X-ray diffraction pattern of the unidentified form is substantially the same as the X-ray powder diffraction pattern of the crystalline form of this invention. If the X-ray powder diffraction pattern is substantially the same as FIG. 1, the previously unknown crystalline form of Sorafenib base can be readily and accurately identified as the crystalline Form-SSB of this invention.

The Sorafenib base crystalline Form-SSB appears to be an anhydrate, which may be evident from the FIG. 3 showing the TGA thermogram. A sample of the crystalline Form-SSB prepared by the inventors had moisture content up to about 0.3% w/w by KF method, which also confirmed the anhydrate nature of the compound. While the invention is not limited to any specific theory, it should be understood however that the crystalline form SSB of Sorafenib base may contain additional residual or unbound moisture without losing its anhydrate character and/or its anhydrate crystalline form-SSB characteristics.

Another embodiment of the present application provides a process for the preparation of Sorafenib tosylate salt, comprising steps of:
a) reacting 4-(4-aminophenoxy)-N-methylpicolinamide with 4-chloro-3-(trifluoro methyl)phenylisocyanate in a high boiling organic solvent at a temperature ranging between 75-90° C., to yield Sorafenib base;
b) combining Sorafenib base with p-toluenesulfonic acid in the presence of methyl ethyl ketone at a temperature below 35° C., to give Sorafenib tosylate.

The individual steps of the process according to the present invention for preparing Sorafenib tosylate salt are detailed separately herein below.

Step a) comprises reacting 4-(4-aminophenoxy)-N-methylpicolinamide (III) with 4-chloro-3-(trifluoromethyl)phenylisocyanate (IV) in a high boiling organic solvent at a temperature ranging from 75-90° C., to yield Sorafenib base (II).

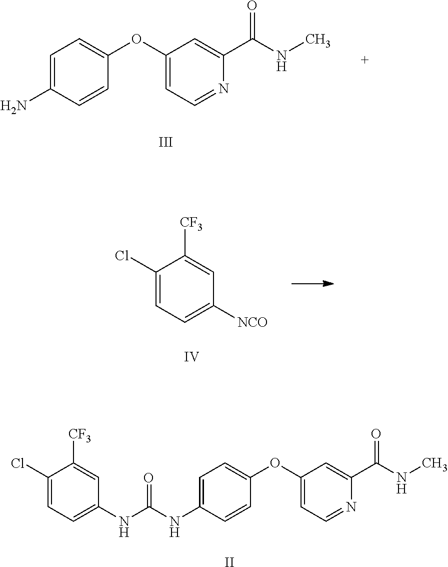

High boiling organic solvent, quantities of different reagents, reaction temperature used, and other relevant details of this step, are similar to the ones described in the previous embodiment related to the preparation of Sorafenib base (M.

Step b) comprises combining Sorafenib base (II) with p-toluenesulfonic acid in the presence of methyl ethyl ketone at a temperature below 35° C., to give Sorafenib tosylate (I).

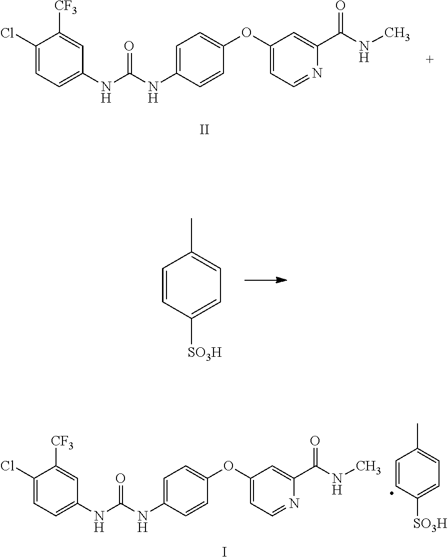

The process for the preparation of Sorafenib tosylate, by reaction of Sorafenib base (II) with p-toluenesulfonic acid in this step, is carried out at an ambient temperature below 35° C., preferably ranging between 20-35° C., but not above 40° C. In view of prior known processes, present invention process in this step, offers significant advantage of preparing Sorafenib tosylate at easily maintainable temperature range which is commercially and industrially viable.

The quantity of the MEK solvent used in this reaction, ranges between 5 to 12 times (in volume) with respect to the weight of Sorafenib material (crude/purified material as obtained from step a) or from any other method known in prior art). In one particular embodiment, for 10 g of Sorafenib, 80 ml of the solvent methyl ethyl ketone was used.

Reaction duration for the present reaction ranges between 2-8 hrs (depending on the actual reaction conditions employed and the progress of the reaction, which is intermittently checked by HPLC). The final product is dried and isolated by conventional methods. The conventional methods for isolating the product may include but are not limited to filtering (with or without vacuum), optionally washing with suitable solvent and drying.

Advantageously, according to the process of the present invention, substantially pure end product is obtained and there is no requirement of any additional purification of final API to remove the impurities so as to bring the final API in compliance with the stipulated regulatory requirements.

In another embodiment of the present application, the present invention provides a one-pot process for the preparation of Sorafenib tosylate (1), comprising the steps of:
a) reacting 4-(4-aminophenoxy)-N-methylpicolinamide (III) with 4-chloro-3-(trifluoromethyl)phenylisocyanate (IV) in a high boiling organic solvent at a temperature ranging between 75-90° C.;

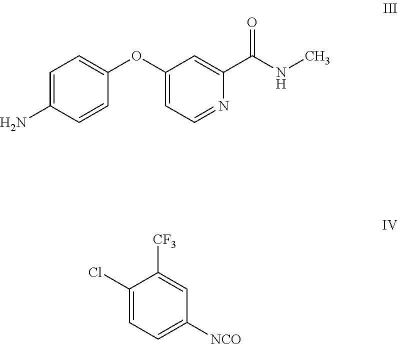

b) adding p-toluenesulfonic acid to the reaction mass of step a) in the presence of methyl ethyl ketone at a temperature below 35° C.;
c) isolating the material as Sorafenib tosylate (I).

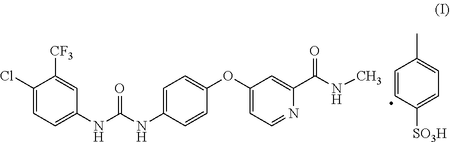

The high boiling organic solvent used for this reaction in step a) may be selected from methyl ethyl ketone (MEK), methyl isobutyl ketone (MIBK) or a mixture thereof.

The compound of formula-I may be isolated by conventional methods and optionally may be dried suitably. The conventional methods for isolating the product may include but are not limited to cooling the reaction mass, wherever required neutralizing, maintaining, filtering (with or without vacuum), optionally washing with suitable solvent and drying.

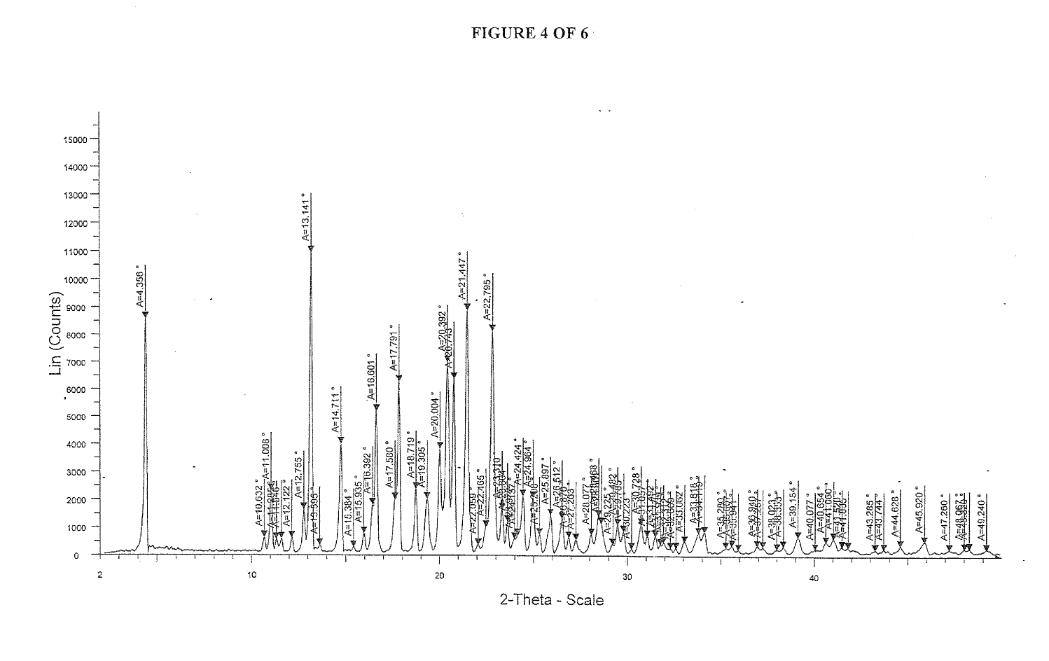

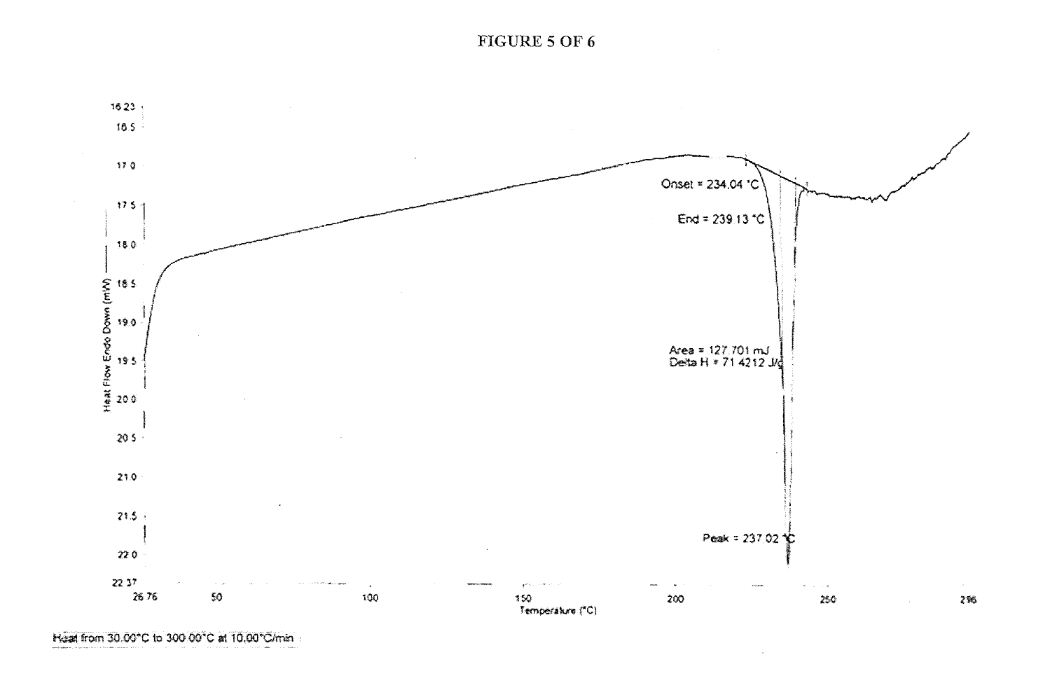

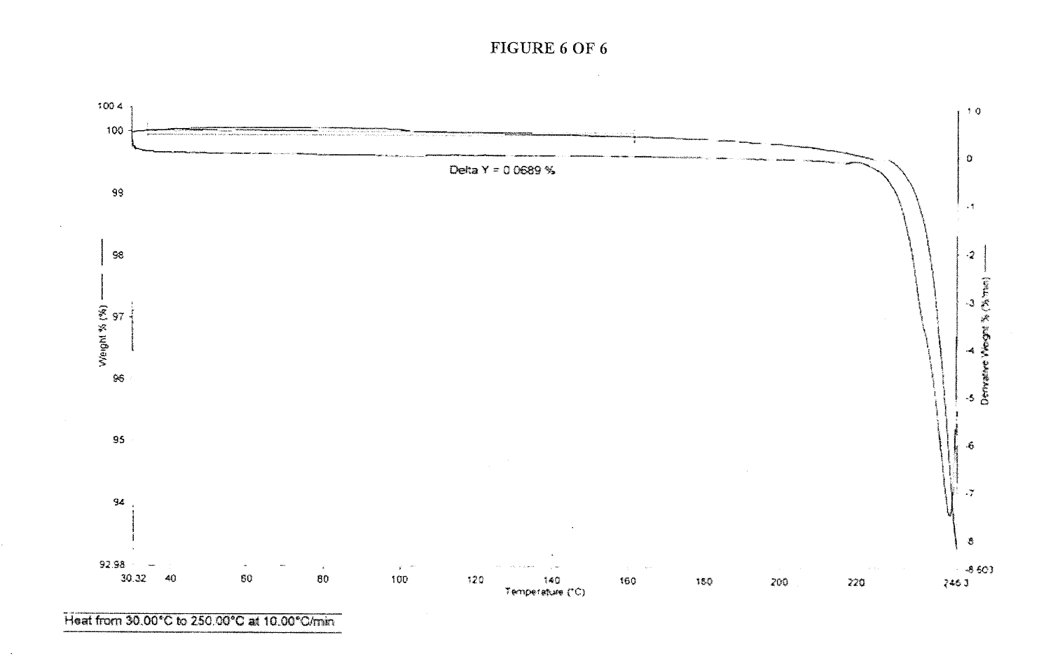

In an embodiment of the present invention, for the processes mentioned herein, Sorafenib tosylate obtained is characterized by PXRD pattern similar to FIG. 4, DSC curve similar to FIG. 5 and TGA spectrum similar to FIG. 6. This analytical data corroborates that Sorafenib tosylate obtained by the invention of present application, is polymorphic Form I of Sorafenib tosylate.

In a further embodiment of the present application, it provides industrially applicable process for direct preparation of crystalline Sorafenib tosylate polymorphic Form I, without any seeding requirement and without isolation of the polymorphic Form II, which are some of the limiting factors for prior disclosed processes. Pure Sorafenib tosylate polymorphic Form I obtained by the processes mentioned herein is substantially free of any other known form of Sorafenib tosylate, for e.g. Form II or Form III etc.

The Sorafenib base crystalline Form-SSB and Sorafenib tosylate Form-I described herein may be characterized by X-ray powder diffraction pattern (XRPD) and Thermal techniques such as differential scanning calorimetry (DSC) analysis. The samples of Sorafenib tosylate Form-I were analyzed by XRPD on a Bruker AXS D8 Advance Diffractometer using X-ray source—Cu Kα radiation using the wavelength 1.5418 Å and lynx Eye detector. DSC was done on a Perkin Elmer Pyris 7.0 instrument. Illustrative examples of analytical data for the Sorafenib base crystalline Form-SSB and Sorafenib tosylate Form-I obtained in the Examples are set forth in the FIGS. 1-6.

In a further embodiment according to the specification, Sorafenib base crystalline Form-SSB may be formulated into a composition, of which at least 95%, by total weight of the Sorafenib in the composition, is the crystalline Form-SSB. In yet another embodiment of the invention, the composition may be substantially free of any other known forms of Sorafenib base whether amorphous or crystalline form.

The Sorafenib base crystalline Form-SSB, may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

In one embodiment of the present invention, it also includes premix comprising one or more pharmaceutically acceptable excipients in the range of 1 to 50% w/w with Sorafenib base crystalline Form-SSB, while retaining the nature of the premix.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Sorafenib base crystalline Form-SSB of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like, Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of Sorafenib base crystalline Form-SSB of the present application may also comprise to include the pharmaceutically acceptable carrier used for the preparation of solid dispersion, wherever utilized in the desired dosage form preparation.

Certain specific aspects and embodiments of the present application will be explained in more detail with reference to the following examples, which are provided by way of illustration only and should not be construed as limiting the scope of the invention in any manner.

### EXAMPLES

### Example 1

### Preparation of Sorafenib Base Crystalline Form-SSB

Charged 60 ml of methyl ethyl ketone at ambient temperature (25-30° C.) in round bottom flask. 20 g of 4-(4-aminophenoxy)-N-methylpicolinamide was added to MEK and stirred for about 15 minutes. Further, 4-chloro-3-trifluoromethylisocyanate (30 g) was added slowly over a period of 5-10 min. Then reaction temperature was raised to 80-85° C., where stirring of the reaction mixture was done for 4-5 h. The reaction mixture was cooled to morn temperature, filtered and washed with toluene. The title product was isolated after drying, as a crystalline material (having XRPD pattern as per FIG. 1; DSC isotherm as per FIG. 2 and TGA thermogram as per FIG. 3).

Yield—75% HPLC purity—99.5%

### Example 2

### Preparation of Crystalline Sorafenib Tosylate

Charged 80 ml of methyl ethyl ketone at room temperature in round bottom flask. Added 10 gm of Sorafenib and stirred for about 15-20 minutes. Slowly added p-toluenesulfonic acid (5.32 g) dissolved in 50 ml MEK. Stirred the reaction mass at 25-30° C. for 4-5 h. Filtered the product and washed with 40 ml of MEK. The title product (having XRPD diffractogram as shown in FIG. 4, DSC curve as shown in FIG. 5 and TGA spectrum as shown in FIG. 6) was isolated after drying.

Yield—95% HPLC purity—99.9%

### Example 3

### One Pot Preparation of Crystalline Sorafenib Tosylate

Charged 60 ml of methyl ethyl ketone at ambient temperature in round bottom flask. Added 20 g of 4-(4-aminophenoxy)-N-methylpicolinamide and stirred for about 15 minutes. Further, 4-chloro-3-trifluoromethylisocyanate (30 g) was added slowly over a period of 5-10 min and the reaction temperature was raised to 80-85° C. Stirred the reaction mixture at this temperature for 4-5 h. Then, the reaction temperature was cooled to room temperature and 80 ml of methyl ethyl ketone was added. Then slowly, p-toluenesulfonic acid (20 g) dissolved in 50 ml MEK was added to the reaction mass which was stirred for 4-5 h. Filtered the product and washed with 120 ml of MEK. The title product was isolated and dried under vacuum at 50-55° C. for 15-17 h.

Yield—88% HPLC purity—99.6%

While the foregoing provides a detailed description of the preferred embodiments of the invention, it is to be understood that the descriptions are illustrative only of the principles of the invention and not limiting. Furthermore, as many changes can be made to the invention without departing from the scope of the invention, it is intended that all material contained herein be interpreted as illustrative of the invention and not in a limiting sense.

We claim:

1. A process for the preparation of crystalline Sorafenib base-Form-SSB, comprising reacting 4-(4-aminophenoxy)-N-methylpicolinamide (III) with 4-chloro-3-(trifluoro methyl)phenylisocyanate (IV) in a high boiling organic solvent at a temperature ranging between 75-90° C., to yield crystalline Sorafenib base-Form-SSB.

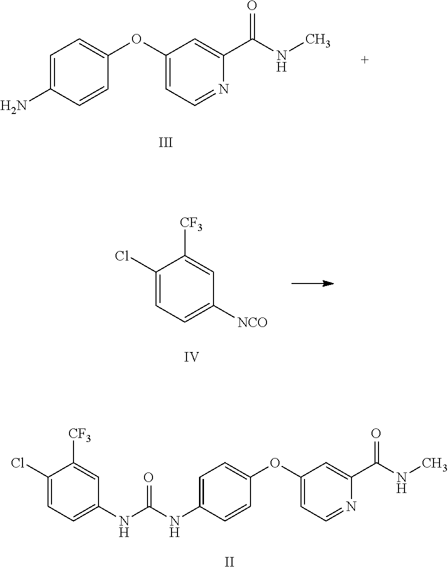

2. A process for the preparation of crystalline Sorafenib base-Form-SSB according to claim 1, wherein the high boiling organic solvent is selected from C₄₋₈ ketones or a mixture thereof.

3. A process for the preparation of crystalline Sorafenib base-Form-SSB according to claim 1, wherein C₄₋₈ ketone is selected from methyl ethyl ketone (MEK) or methyl isobutyl ketone (MIBK).

4. A process for the preparation of crystalline Sorafenib base-Form-SSB according to claim 1, wherein reaction of 4-(4-aminophenoxy)-N-methylpicolinamide (III) with 4-chloro-3-(trifluoromethyl)phenylisocyanate (IV), is carried out at temperature ranging between 75-90° C.

5. A process for the preparation of crystalline Sorafenib base-Form-SSB according to claim 1, wherein crystalline Sorafenib base-Form-SSB obtained is characterized by X-ray powder diffraction pattern substantially according to FIG. 1 and DSC isothermal pattern substantially according to FIG. 2.

6. Crystalline Sorafenib base-Form-SSB, characterized by X-ray powder diffraction pattern comprising at least 5 characteristic 2θ° peaks selected from the XRPD peak set of 9.9, 11.4, 12.6, 14.6, 15.2, 15.6, 18.1, 18.6, 21.8, 22.5, 22.9, 23.6, 24.8, 25.2±0.20 2θ° and DSC isotherm comprising a single endothermic peak ranging between 202 to 212° C.

7. Crystalline Sorafenib base-Form-SSB according to claim 6, characterized by X-ray powder diffraction pattern substantially according to FIG. 1 and DSC isothermal pattern substantially according to FIG. 2.

8. A process for the preparation of Sorafenib tosylate using crystalline Sorafenib base-Form-SSB, comprising steps of:
a) reacting 4-(4-aminophenoxy)-N-methylpicolinamide (III) with 4-chloro-3-(trifluoromethyl)phenylisocyanate (IV) in a high boiling organic solvent at a temperature ranging between 75-90° C., to yield crystalline Sorafenib base (II)-Form-SSB; and 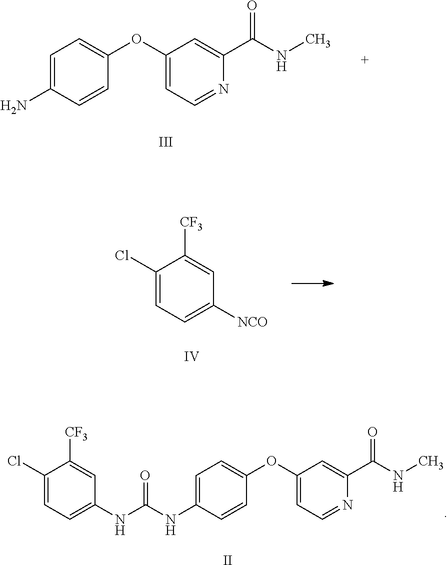
b) combining crystalline Sorafenib base (II)-Form-SSB with p-toluenesulfonic acid in the presence of methyl ethyl ketone at a temperature below 35° C., to give Sorafenib tosylate (I).

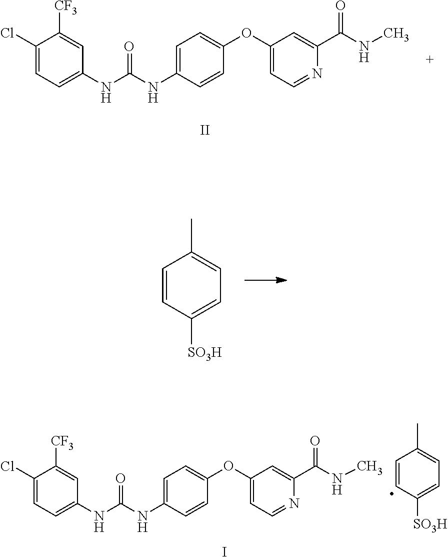

9. A process for the preparation of Sorafenib tosylate using crystalline Sorafenib base-Form-SSB according to claim 8, wherein the high boiling organic solvent is selected from C₄₋₈ ketones or a mixture thereof.

10. A process for the preparation of Sorafenib tosylate using crystalline Sorafenib base-Form-SSB according to claim 8, wherein C₄₋₈ ketone is selected from methyl ethyl ketone (MEK) or methyl isobutyl ketone (MIBK).

11. A process for the preparation of Sorafenib tosylate using crystalline Sorafenib base-Form-SSB according to claim 8, wherein reaction of 4-(4-aminophenoxy)-N-methylpicolinamide (III) with 4-chloro-3-(trifluoromethyl)phenylisocyanate (IV), is carried out at temperature ranging between 75-90° C.

12. A process for the preparation of Sorafenib tosylate using crystalline Sorafenib base-Form-SSB according to claim 8, wherein the obtained final product is crystalline Sorafenib tosylate Form-I characterized by X-ray powder diffraction pattern substantially according to FIG. 4.

13. A pharmaceutical composition comprising crystalline Sorafenib base-Form-SSB according to claim 6, together with one or more pharmaceutically acceptable excipients.
